(19) United States
(12) Patent Application Publication
(10) Pub. No.: US 2009/0035301 A1
(43) Pub. Date: Feb. 5, 2009

(54) METHODS FOR DIAGNOSING AND TREATING BLADDER CANCER

(75) Inventors: Katherine Meyer Siegler, Seminole, FL (US); Pedro L. Vera, Largo, FL (US)

Correspondence Address: BLANK ROME LLP, 600 NEW HAMPSHIRE AVENUE, N.W., WASHINGTON, DC, 20037, US

(73) Assignee: The United States of America Department of Veterans Affairs, Washington, DC (US)

(21) Appl. No.: 12/183,687

(22) Filed: Jul. 31, 2008

Related U.S. Application Data
(63) Continuation of application No. 11/064,955, filed on Feb. 25, 2005, now abandoned.
(60) Provisional application No. 60/547,052, filed on Feb. 25, 2004.

(51) Int. Cl. A61K 39/395; A61P 35/00; A61K 31/715; A61K 31/7088

(52) U.S. Cl. 424/130.1; 514/54; 514/44

(57) ABSTRACT

The present invention relates to the diagnosis and treatment of bladder cancer. More specifically, this invention uses the levels of macrophage migration inhibitory factor (MIF) produced by the bladder epithelia (urothelia) as a marker for bladder cancer. Moreover, the present invention also provides a method for attenuating bladder carcinoma by inhibiting of macrophage MIF.

This application claims priority to U.S. Provisional Patent Application Ser. No. 60/547,052, filed Feb. 25, 2004, which is incorporated herein by reference.

### FIELD OF THE INVENTION

The present invention relates to the diagnosis and treatment of bladder cancer. More specifically, this invention uses the levels of macrophage migration inhibitory factor (MIF) produced by the bladder epithelia (urothelia) as a marker for bladder cancer. Moreover, inhibition of macrophage MIF is effective in attenuating bladder carcinomas.

### BACKGROUND OF THE INVENTION

According to 2003 estimates, urinary bladder cancer will be diagnosed in 57,400 Americans and will result in 12,500 deaths (Jema et al., CA Cancer J. Clin. 2003, 53:5-26). Of these new cases, 80 to 90% will originally present as tumors of the epithelium or submucosa, with the majority being transitional cell carcinomas (Small et al., Cancer 2003, 97: 2090-2098; and Piazza et al., Cancer Res. 2001, 61:3961-3968). Transurethral resection of bladder tumor remains the initial line of defense in treatment of superficial bladder cancer. However, this treatment is hardly adequate as the recurrence rate in treated patients approaches 50 to 70% and 5 to 40% of recurrent cancers progress (Small et al.; and Herr et al., J. Urol. 1989, 141: 22-29). In an attempt to curb the reoccurrence rate, a variety of immunotherapies and chemotherapies have been devised, with the most common being intravesical bacillus Calmette-Guerin. The high rate of mortality associated with invasive urinary bladder cancer and the high incidence of reoccurrence after treatment demonstrate the need for a better understanding of bladder cancer and new therapeutic agents for treatment.

Cystoscopy and biopsy are the standards for the diagnosis of bladder cancer. However, inherent in this modality is the inability to detect small cancers. In addition, this procedure is expensive and uncomfortable to patients. Because of this, several non-invasive methods have been developed including voided urine cytology (low sensitivity) and the detection of various urine markers including nuclear matrix protein-22 and HA. The urine markers are often used in conjunction with voided urine cytology to improve sensitivity.

MIF was first described thirty years ago and was designated as a cytokine, a chemical mediator, which regulates cell growth by inducing the expression of specific target genes. The initial described function of MIF was as a regulator of inflammation and immunity. It is expressed in the brain, and eye lens, is a delayed early response gene in fibroblasts, and it has been reported that this protein can be found in prostate tissues. MIF has been shown to be a pituitary, as well as macrophage cytokine and a critical mediator of septic shock. Recent studies also suggest that MIF may have an autocrine function for embryo development and is produced by the Leydig cells of the testes. Thus, it appears that this cytokine may play a fundamental role in cell growth regulation and possibly development.

U.S. Pat. No. 6,043,044 discloses the use of prostate tissue extracts as a patient sample to determine the amount of MIF. Immuno- and RNA blot analysis performed using homogenized tissue that contains variable proportions of epithelial and stromal cells still determined significant differences in the levels of MIF protein produced by metastatic tissue (490.3+/−71.3 ng/mg total protein).

### SUMMARY OF THE INVENTION

This application relates to the diagnosis, prognosis, and treatment of bladder cancer. Bladder epithelial carcinoma produces an increased level of macrophage migration inhibitory factor (MIF). The MIF is secreted from the cells into the bladder as a feed-back mechanism in tumor growth. Because of the secretion, the MIF levels can be determined in the urine or through a bladder biopsy.

The present invention provides methods for detecting or diagnosing or prognosticating bladder cancer. The methods comprise determining the levels of MIF produced by bladder epithelia. The method can measure the secreted MIF from a urine sample, or the intracellular MIF from a bladder biopsy.

The present invention further provides methods for monitoring the treatment of an individual with bladder cancer. The methods comprise administering a pharmaceutical composition to an individual and determining the levels of MIF produced by bladder epithelia.

The present invention further provides methods for screening for an agent capable of modulating the onset or progression of bladder cancer. The methods comprise exposing an individual to the agent and determining the levels of MIF produced by bladder epithelia.

In embodiments of the present invention, levels of MIF are determined by detecting MIF gene product in the bladder cells or urine using immunoassays or nucleic acid analysis, preferably mRNA. Gene products as recited herein can be nucleic acid (DNA or RNA) and/or proteins. In the case of DNA and RNA, detection occurs through hybridization with oligonucleotide probes. In the case of proteins, detection occurs though various protein interaction. Because MIF in urine is measured, the present invention provides a non-invasive test for bladder cancer.

In addition, the present invention provides methods for treating bladder cancer by inhibiting macrophage MIF. Because macrophage MIF acts in a feedback mechanism to allow proliferation of cancer cells, inhibition anywhere along the mechanism can be effected to attenuate tumor growth. MIF can be inhibited by, but is not limited to, anti-MIF antibodies, MIF-antagonists, hylaluronan, anti-sense MIF oligonucleotides, or combinations thereof.

### BRIEF DESCRIPTION OF THE FIGURES

FIG. 1 shows localization of MIF in the human bladder and prostate—A) MIF protein in the normal human bladder; B) MIF protein in the normal human prostate; C) MIF mRNA in the human bladder; D) MIF mRNA in the human prostate.

FIG. 2 shows cell growth of bladder HT-1376 cells when treated with HA, αMIF, and anti-sense MIF oligonucleotides.

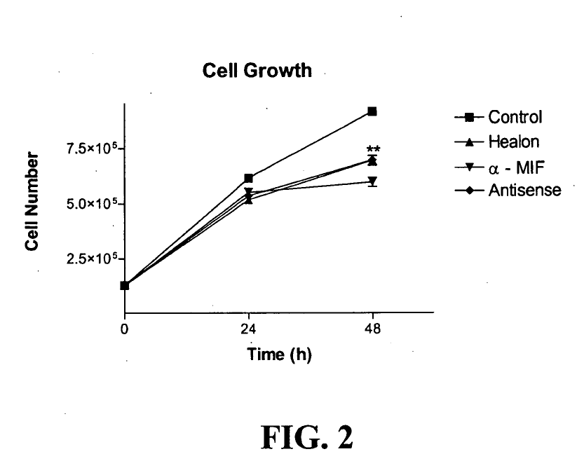

FIG. 3 shows caspase-3 activity within the cell lysates treated with HA, αMIF, and anti-sense MIF oligonucleotides.

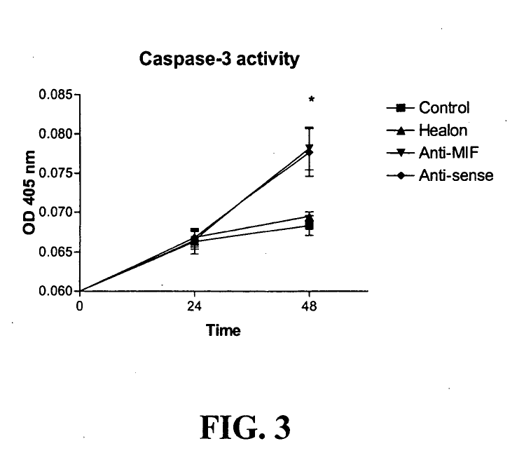

FIG. 4 shows secreted (A) and intracellular (B) MIF in bladder HT-1376 cells treated with HA, αMIF, and anti-sense MIF oligonucleotides.

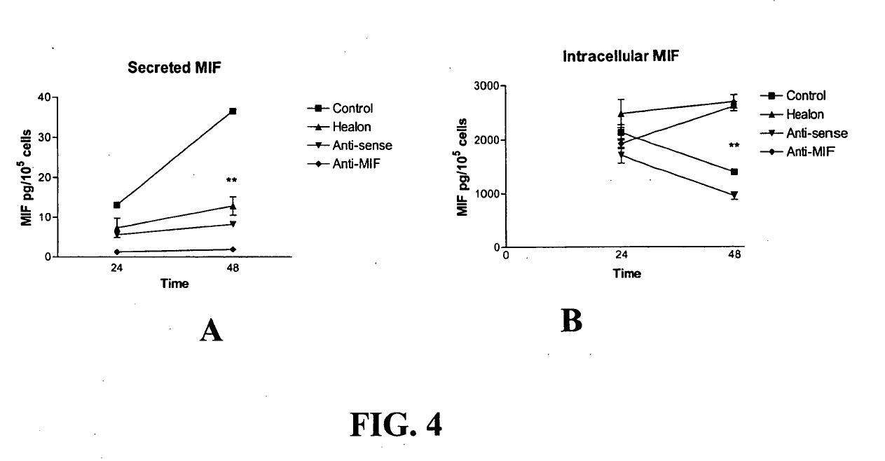

FIG. 5 shows MIF mRNA in HT-1376 cells treated with HA, αMIF, and anti-sense MIF oligonucleotides.

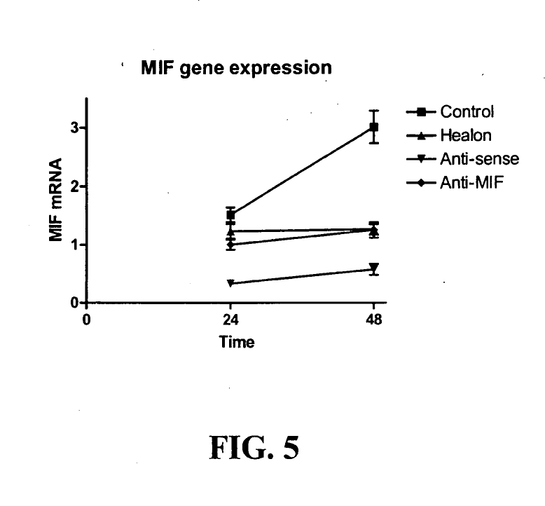

### DESCRIPTION OF THE PREFERRED EMBODIMENT

Many biological functions are accomplished by altering the expression of various genes through transcriptional (e.g., through control of initiation, provision of RNA precursors, RNA processing, etc.) and/or translational control. For example, fundamental biological processes such as cell cycle, cell differentiation and cell death, are often characterized by the variations in the expression levels of individual gene or group of genes.

Changes in gene expression also are associated with pathogenesis. For example, the lack of sufficient expression of functional tumor suppressor genes and/or the over expression of oncogene/protooncogenes could lead to tumorgenesis or hyperplastic growth of cells (Marshall (1991) Cell 64:313-326; Weirlberg (1991), Science 254:1138-1146). Thus, changes in the expression levels of particular gene or group of genes (e.g., oncogenes or tumor suppressors) serve as signposts for the presence and progression of various diseases.

Monitoring changes in gene expression may also provide certain advantages during drug screening development. Often drugs are screened and prescreened for the ability to interact with a major target without regard to other effects the drugs have on cells. Often such other effects cause toxicity in the whole animal, which prevent the development and use of the potential drug.

The present inventors have identified MIF as a gene marker associated with bladder cancer. Changes levels of MIF production by bladder epithelia can also provide useful markers for diagnostic uses as well as markers that can be used to monitor disease states, disease progression, drug toxicity, drug efficacy and drug metabolism. Further, the present inventors have also discovered that inhibiting MIF is effective in attenuating tumor growth in bladder cancer.

### Use of MIF as Diagnostics

As described herein, the MIF levels may be used as diagnostic markers for the prediction or identification of bladder cancer. For instance, a urine or bladder biopsy sample from a patient may be assayed by any of the methods described herein or by any other method known to those skilled in the art, and the expression levels of MIF may be compared to the expression levels found in normal individuals or in cancer patients. The expression levels of MIF that substantially resemble an expression level of normal or of diseased bladder may be used, for instance, to aid in disease diagnosis and/or prognosis. Comparison of the MIF levels may be done by researcher or diagnostician or may be done with the aid of a computer and databases.

### Use of MIF for Drug Screening

According to the present invention, MIF levels may be used as markers to evaluate the effects of a candidate drug or agent on a bladder cancer patient.

A patient is treated with a drug candidate and the progression of bladder cancer is monitored over time. This method comprises treating the patient with an agent, obtaining a sample from the patient, determining levels of MIF produced by bladder epithelia, and comparing the levels of MIF over time to determine the effect of the agent on the progression of bladder cancer.

The candidate drugs or agents of the present invention can be, but are not limited to, peptides, small molecules, vitamin derivatives, as well as carbohydrates. Dominant negative proteins, DNA encoding these proteins, antibodies to these proteins, peptide fragments of these proteins or mimics of these proteins may be introduced into the patient to affect function. “Mimic” as used herein refers to the modification of a region or several regions of a peptide molecule to provide a structure chemically different from the parent peptide but topographically and functionally similar to the parent peptide (see Grant (1995), in Molecular Biology and Biotechnology, Meyers (editor) VCH Publishers). A skilled artisan can readily recognize that there is no limit as to the structural nature of the candidate drugs or agents of the present invention.

### Use of MIF for Monitoring Disease Progression

As described above, the expression of MIF may also be used as markers for the monitoring of disease progression, for instance, the development of bladder cancer. For instance, a sample from a patient may be assayed by any of the methods described herein; and the expression levels of MIF in the sample may be compared to the expression levels found in normal individuals. The MIF levels can be monitored over time to track progression of the disease. Comparison of the MIF levels may be done by researcher or diagnostician or may be done with the aid of a computer and databases.

### Assay Formats

The over expression of MIF is manifest at both the level of messenger ribonucleic acid (mRNA) and protein. It has been found that increased MIF, determined by either mRNA levels or biochemical measurement of protein levels using immunoassays, is associated with bladder cancer.

In an embodiment of the present invention, MIF levels are detected by immunoassays. Generally, immunoassays involve the binding of the MIF and anti-MIF antibody. The presence and amount of binding indicate the presence and amount of MIF present in the sample. Examples of immunoassays include, but are not limited to, ELISAs, radioimmunoassays, and immunoblots, which are well known in the art. The antibody can be polyclonal or monoclonal and is preferably labeled for easy detection. The labels can be, but are not limited to biotin, fluorescent molecules, radioactive molecules, chromogenic substrates, chemi-luminescence, and enzymes.

In a preferred embodiment, ELISA, based on the capture of MIF by immobilized monoclonal anti-MIF antibody followed by detection with biotinylated polyclonal anti-MIF antibody, is used to detect MIF. In this system, the wells of a multi-well plate are coated with the monoclonal antibody and blocked with milk (albumin blocking should be avoided because MIF has been shown to bind albumin). Tissue or urine samples are then added to the wells and incubated for capture of MIF by the monoclonal antibody. The plate is then detected with the polyclonal antibody and strepavidine-alkaline phosphatase conjugate.

In another embodiment, MIF levels are detected by measuring nucleic acid levels in the bladder tissue or urine, preferably MIF mRNA. This is accomplished by hybridizing the nucleic acid in the sample with oligonucleotide probes that is specific for the MIF gene.

Nucleic acid samples used in the methods and assays of the present invention may be prepared by any available method or process. Methods of isolating total RNA are also well known to those of skill in the art. For example, methods of isolation and purification of nucleic acids are described in detail in Chapter 3 of Laboratory Techniques in Biochemistry and Molecular Biology: Hybridization With Nucleic Acid Probes, Part I—Theory and Nucleic Acid Preparation, Tijssen, (1993) (editor) Elsevier Press. Such samples include RNA samples, but also include cDNA synthesized from a mRNA sample isolated from a cell or tissue of interest. Such samples also include DNA amplified from the cDNA, and an RNA transcribed from the amplified DNA. One of skill in the art would appreciate that it is desirable to inhibit or destroy RNase present in homogenates before homogenates can be used.

Nucleic acid hybridization simply involves contacting a probe and target nucleic acid under conditions where the probe and its complementary target can form stable hybrid duplexes through complementary base pairing (see U.S. Pat. No. 6,333,155 to Lockhart et al, which is incorporated herein by reference). Methods of nucleic acid hybridization are well known in the art. In a preferred embodiment, the probes are immobilized on solid supports such as beads, microarrays, or gene chips.

The hybridized nucleic acids are typically detected by detecting one or more labels attached to the sample nucleic acids and or the probes. The labels may be incorporated by any of a number of means well known to those of skill in the art (see U.S. Pat. No. 6,333,155 to Lockhart et al, which is incorporated herein by reference). Commonly employed labels include, but are not limited to, biotin, fluorescent molecules, radioactive molecules, chromogenic substrates, chemiluminescent labels, enzymes, and the like. The methods for biotinylating nucleic acids are well known in the art, as are methods for introducing fluorescent molecules and radioactive molecules into oligonucleotides and nucleotides.

Detection methods, for both the immunoassays and the nucleic acid assays, are well known for fluorescent, radioactive, chemiluminescent, chromogenic labels, as well as other commonly used labels. Briefly, fluorescent labels can be identified and quantified most directly by their absorption and fluorescence emission wavelengths and intensity. A microscope/camera setup using a light source of the appropriate wavelength is a convenient means for detecting fluorescent label. Radioactive labels may be visualized by standard autoradiography, phosphor image analysis or CCD detector. Other detection systems are available and known in the art.

### MIF Inhibition

The treatment of bladder cancer involves inhibition of MIF. Because MIF is involved in a cell signaling loop, the inhibition of MIF anywhere along the loop is appropriate for the present invention. This includes inhibition of intracellular MIF production and/or MIF excretion. Compounds used to inhibit MIF can be, but is not limited to, MIF antibodies, hyaluronan (HA), MIF anti-sense oligonucleotides, or MIF specific blockers.

Compounds to inhibit MIF can be delivered using intravesical therapies. Intravesical therapies are treatments that are administered directly into the bladder by means of a catheter. These therapies are routine for various bladder disorders including bladder cancer. In an attempt to curb the recurrence rate a variety of immunotherapies and chemotherapies have been formulated, with the most common being intravesical Bacillus Calmette-Guerin (BCG). Although the combination of transurethral resection bladder tumors and BCG therapy have been of modest improvement, a 10 year follow up study showed that only 31% of patients suffered no recurrence and in 41% of patients the cancer progressed. Various experimental therapies based upon the use of targeted monoclonal antibodies are currently in clinical trials for patients with bladder cancer (anti-HER2 monoclonal antibodies (Herceptin(R), EGFR targeted agents (IMC-C225 Cetuximab(R), ZD1389 Iressa(R), OSI-774 Tarceva(R), GW 57016). It is expected that these monoclonal antibody treatments will supplement current chemotherapeutic drugs regimens. MIF inhibition can, thus, be used alone or to supplement current treatments.

Without further description, it is believed that one of ordinary skill in the art can, using the preceding description and the following illustrative examples, make and utilize the compounds of the present invention and practice the claimed methods. The following example is given to illustrate the present invention. It should be understood that the invention is not to be limited to the specific conditions or details described in this example.

### Example 1

### Human Bladder Adenocarcinoma Cells Synthesize and Secrete MIF in vitro

In order to establish the relevance of MIF to urogenital disease in humans we sought to determine if human bladder epithelial cells secrete MIF. Human bladder HT-1376 cells (ATCC, Manasas, Va.) were cultured for 48 hours and the culture medium was assayed for MIF using ELISA. In addition, intracellular content of MIF was assayed from cell lysates. These human bladder adenocarcinoma cells synthesize MIF (226±0.6 μg/mg protein). The average MIF concentration secreted into the medium by these cells was 15 ng/ml. Expression of MIF was confirmed by RT-PCR analysis. Thus establishing MIF secretion by human bladder epithelia.

### Example 2

### IHC/insitu

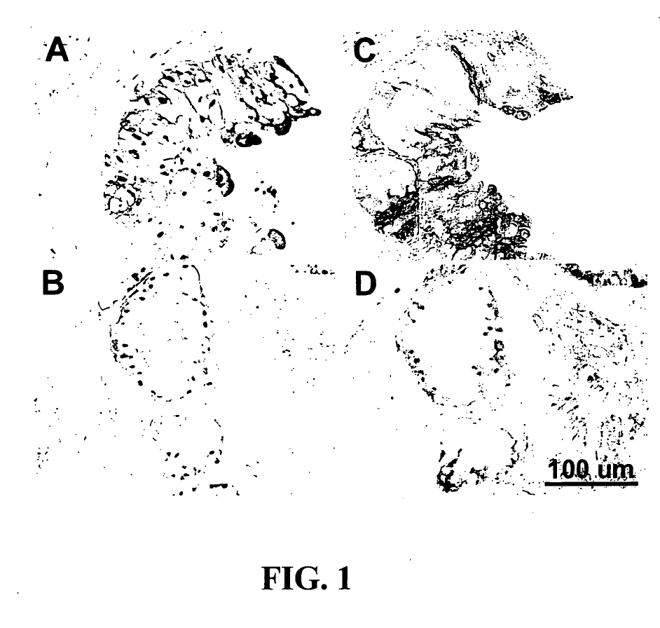

The intracellular location of MIF protein and mRNA in the normal human bladder and prostate was determined by immunohistochemistry and in situ hybridization. Four-micron thick sections were processed for immunohistochemistry using an anti-human MIF antibody (R&D Systems, same antibody that is used for detection in the ELISA) and a standard peroxidase-antiperoxidase protocol. The location of MIF mRNA within these tissues was determined by insitu hybridization. MIF specific templates are prepared by reverse transcription of total RNA from a human bladder cell line (HT-1376) followed by amplification of a MIF 254 bp fragment (nucleotides 67-321). T7 RNA polymerase promoters were ligated to the resulting PCR product and biotinylated single stranded RNA oligonucleotide probes prepared from this template by in vitro transcription. Paraffin embedded tissue is prepared for hybridization by dewaxing through xylene, followed by hydration through alcohol and Proteinase K digestion. 100 ng biotin-labeled anti-sense probe was added to tissue and allowed to hybridize overnight at 37° C. Following hybridization, tissue was extensively washed with TBS-T, then washed with increasing stringency using 0.2×SSC/0.05% Tween-20. Endogenous peroxidase activity was quenched by treatment with 3% hydrogen peroxide. Following blocking the biotinylated probes were detected by incubation with an anti-avidin-biotin conjugate, followed by avidin-peroxidase (Sigma, St. Louis, Mo.). The diaminobenzidine substrate forms a colored precipitate product that is detected by light microscopy. Normal human bladder urothelium exhibits intense staining for MIF protein (see FIG. 1).

### Example 3

### Intravesical MIF Blockage Alters Bladder Cancer Cell Growth and Cytokine Expression

Bladder transitional cell carcinoma cells secrete MIF into the culture medium, therefore it is possible that MIF release is participating in a regulatory loop that amplifies or maintains bladder cancer cell growth. We determined whether it was possible to disrupt this loop by neutralizing MIF. Preliminary data from experiments performed on rats determined that intravesical MIF antibody reversed changes in gene expression (Disclosure VA IP 03-079). In addition intravesical MIF antibody reduced histological signs of bladder inflammation. Our hypothesis is that MIF may be involved in the initiation or the continuation of inflammatory and growth-promoting processes, which are essential to tumor growth; therefore, blockade of MIF may be able to prevent or reverse bladder cancer.

In these experiments we chose three different mechanisms of MIF inhibition. A non-specific MIF inhibitor, Healon (hyaluronan, HA), which prevents MIF signal transduction by binding to the cell surface receptor CD44, anti-MIF monoclonal antibodies (αMIF), and anti-sense MIF oligonucleotides (ASO). Previously, MIF was reported to bind to HA. HA is a nonsulfated linear glycosaminoglycan that consists of repeating disaccharide units of D-glucuronic acid and N-acetyl-glucosamine, which binds to the cell adhesion molecule CD44. HA functions in a number of physiological events including cell adhesion and proliferation. Recent studies have determined that binding of large molecular weight hyaluronan to CD44 inhibits CD44 cleavage, which is necessary for CD44 induced angiogenesis and tumor promotion. Large HA polymers bind to CD44 with greater avidity that smaller HA oligosaccharides, therefore intravesical HA is a potential therapy for bladder carcinoma. Anti-MIF antibodies have proven to be potent as anti-tumor treatments in animal models. Intravesical anti-MIF antibodies are therefore a potential new treatment for bladder cancer.

### Cell Growth

The effect of treatment with MIF inhibitors on cell proliferation was assessed 24 and 48 hours after initiation of treatment. Human bladder HT-1376 cells (ATCC, Manasas, Va.) were plated in 96 well plates at starting density of 2,500 cells/cm². A significant decrease in cell numbers was seen at 48 h with all MIF inhibitor treatments (ANOVA, Newman-Keuls, p<0.01). Therefore, treatment with MIF inhibitors slows bladder cancer cell proliferation.

### Apoptosis

The reduction in cell proliferation seen with MIF inhibitor treatment could possibly be due to induction of apoptosis. To assess this we determined caspase-3 activity within the cell lysates of 48h cultures. Caspase-3 activity assay determined a slight but significant increase in activity in 48h MIF anti-sense and MIF antibody treated cultures, which could account in part for the decrease in cell numbers (ANOVA, Newman-Keuls, p<0.05). There was no significant increase in caspase-3 activity in the HA treated cultures This suggests that apoptosis induction was only partially responsible for the observed decrease in cell proliferation.

### MIF Protein Synthesis and Secretion

Next, we determined if addition of MIF inhibitors in the culture media changed the synthesis or release of MIF by these cells. In vitro treatment of HT-1376 bladder epithelial cells with MIF ASO resulted in a 50% decrease in cellular MIF content along with a 4-fold decrease in secreted MIF, suggesting that ASO treatment inhibited MIF synthesis. Similar treatment of HT-1376 cells with HA resulted in a 2-fold increase in cellular MIF content along with a 2.8 fold decrease in secreted MIF, suggesting that this treatment prevented MIF release from the cells. Treatment with αMIF resulted in a 19-fold decrease in detectable extracellular MIF concomitant with a 35% decrease in cellular MIF content, suggesting that αMIF also inhibited MIF production.

### Gene Expression

The next analysis determined if MIF inhibitor treatments attenuated MIF gene expression. Total RNA was extracted from tissue, reverse transcribed and the resulting reaction used for polymerase chain reaction (PCR). PCR conditions for MIF were detailed by Vera et al. (J. Urol. 2003, 170:623-627). Gene specific PCR products were normalized to an 18S rRNA internal standard (Ambion) by determining gene expression ratio (area-intensity of MIF specific band divided by area-intensity of 18S rRNA band). All forms of MIF inhibition resulted in a significant decrease in MIF mRNA. HA and αMIF treatment resulted in a greater than 2-fold decrease in MIF mRNA, while ASO treatment resulted in a 6-fold decrease in MIF mRNA amounts. Therefore, the affects of MIF inhibition seen in HT-1376 cells were due in part to a reduction in MIF mRNA.

### Cytokine Secretion

MIF plays a key role in pro-inflammatory gene and cytokine expression. Therefore changes in MIF expression or protein content are likely to affect the expression of additional cytokines. This may in fact be a key in the utility of MIF inhibition in treating bladder adenocarcinoma. To test this, cytokine expression changes induced by MIF inhibition were also evaluated by cytokine protein array analysis (RayBiotech, Norcross, Ga.), which detected secreted cytokines. MIF inhibition resulted in reduction in the secreted amounts of several cytokines, notable among these were MIF, TNF-α and TGF-β3. A summary of the cytokine secretion changes (2 fold or greater) seen with MIF inhibition is described in Table 1.

TABLE 1

| | | Treatment | | |
|---|---|---|---|---|
| Secreted Cytokines | | Healon | α-MIF | Anti-sense |
| 1 | ENA-78 | | Not detected | Not detected |
| 2 | GRO | | Not detected | Not detected |
| 3 | GRO-α | | Not detected | Not detected |
| 4 | IL-1α | | Not detected | Not detected |
| 5 | IL-1β | | Not detected | Not detected |
| 6 | IL-2 | | ↑ 8.0 | Not detected |
| 7 | IL-8 | | Not detected | |
| 8 | IL-12 | | Not detected | Not detected |
| 9 | IL-15 | ↓ 2.7 | Not detected | Not detected |
| 10 | IFN-γ | | Not detected | Not detected |
| 11 | MCP-1 | | Not detected | |
| 12 | MCP-2 | ↓ 4.4 | Not detected | Not detected |
| 13 | MCP-3 | ↓ 3.3 | Not detected | ↓ 25 |
| 14 | MCSF | | Not detected | Not detected |
| 15 | MDC | | Not detected | ↑ 2.5 |
| 16 | MIG | | Not detected | Not detected |
| 17 | MIP-1β | | Not detected | Not detected |
| 18 | MIP-1 delta | ↓ 50.0 | Not detected | |
| 19 | RANTES | ↓ 4.4 | Not detected | Not detected |
| 20 | SCF | | Not detected | Not detected |
| 21 | SDF-1 | | Not detected | Not detected |
| 22 | TARC | ↓ 2.2 | Not detected | Not detected |
| 23 | TNF-α | Not detected | Not detected | ↓ 4.3 |
| 24 | EGF | | Not detected | ↓ 2.0 |
| 25 | IGF-1 | Not detected | Not detected | Not detected |
| 26 | Ang | Not detected | Not detected | Not detected |
| 27 | OSM | | Not detected | Not detected |
| 28 | Tpo | | | Not detected |
| 29 | VEGF | | Not detected | |
| 30 | PDGF-B | | Not detected | Not detected |
| 31 | Leptin | ↓ 45.0 | | Not detected |
| 32 | BDNF | | Not detected | ↓ 3.1 |
| 33 | BLC | ↓ 50.0 | Not detected | ↓ 3.0 |
| 34 | Ckβ 8-1 | Not detected | Not detected | Not detected |
| 35 | Eotaxin | Not detected | Not detected | Not detected |
| 36 | Eotaxin-2 | ↓ 6.8 | Not detected | ↑ 3.3 |
| 37 | Eotaxin-3 | Not detected | Not detected | Not detected |
| 38 | FGF-4 | | Not detected | Not detected |
| 38 | FGF-6 | ↓ 2.6 | | ↓ 2.9 |
| 40 | FGF-9 | | ↑ 12.5 | |
| 41 | Fit-3 Ligand | ↓ 50.0 | ↑ 36.5 | Not detected |
| 42 | Fractalkine | | | Not detected |
| 43 | GCP-2 | ↓ 3.1 | | ↓ 3.1 |
| 44 | GDNF | | Not detected | ↓ 3.0 |
| 45 | HGF | ↓ 12.5 | | Not detected |
| 46 | IGFBP-1 | | | |
| 47 | IGFBP-2 | | Not detected | ↓ 3.3 |
| 48 | IGFBP-3 | | Not detected | |
| 49 | IGFBP-4 | ↓ 60.0 | Not detected | ↓ 2.5 |
| 50 | IL-16 | ↓ 2.1 | ↓ 2.6 | ↓ 2.5 |
| 51 | IP-10 | | | |
| 52 | LIF | | Not detected | ↓ 2.8 |
| 53 | LIGHT | | Not detected | Not detected |
| 54 | MIF | Not detected | Not detected | Not detected |
| 55 | MIP-3α | Not detected | Not detected | ↓ 61.0 |
| 56 | NAP-2 | | Not detected | ↓ 3.6 |
| 57 | NT-3 | ↓ 2.1 | Not detected | ↓ 2.3 |
| 58 | NT-4 | | ↓ 3.5 | Not detected |
| 59 | osteoprotegrin | | Not detected | ↓ 3.6 |
| 60 | PARC | ↓ 2.4 | Not detected | ↓ 3.9 |
| 61 | PIGF | | ↓ 2.1 | Not detected |
| 62 | TGF β-2 | | Not detected | ↓ 4.7 |
| 63 | TGF β-3 | ↓ 32.0 | | Not detected |
| 64 | TIMP-1 | ↓ 2.6 | | ↓ 2.8 |
| 65 | TIMP-2 | | Not detected | ↓ 3.8 |

HT-1376 cells express and secrete 65 of the 79 cytokines present on the array. The three types of MIF inhibition all affected the cytokine secretion patterns of these cells. In table 1, blank cells indicate no difference between the amounts of cytokine secreted by the treated and control cells. Arrows indicate an increase or decrease in the fold amount of cytokine secreted. Not detected indicates that the cytokine was no longer detectable following MIF inhibition. Healon decreased the secreted amount of 27 cytokines. TNF-α and MIF were not detected with Healon treatment, however there was no affect on IL-1β. αMIF decreased the amounts of 55 secreted cytokines with no detectable amounts of TNF-α, MIF and IL-1β. MIF ASO treatment decreased the amounts of 71 secreted cytokines and again TNF-α, MIF and IL-1β were not detected. The MIF inhibitors tested did not affect IGF BP-1 or IP-10.

In vitro treatment of HT-1376 bladder epithelial cells with MIF inhibitors resulted in a 40% decrease in cell growth. MIF antibody treatment resulted in an 87% increase in cellular MIF content along with an 18-fold decrease in secreted MIF, suggesting that MIF antibody treatment inhibited MIF release. AS treatment resulted in a 50% decrease in cellular MIF content along with a 4-fold decrease in secreted MIF, suggesting that AS treatment inhibited MIF synthesis. Similar treatment of HT-1376 cells with Healon resulted in a 2-fold increase in cellular MIF content along with a 2.8 fold decrease in secreted MIF, suggesting that this treatment prevented MIF release for the cells. MIF inhibition is a potential avenue of therapy in bladder cancer.

### Example 4

### ELISA

A human MIF-specific “sandwich” ELISA technique was developed, based on the capture of MIF by immobilized monoclonal antibody (MAB289, R&D Systems) followed by detection with goat polyclonal anti-MIF affinity purified IgG (BAF289, R&D Systems). This assay was successfully used previously to detect MIF levels in serum and urine. This is another application of this protocol in which MIF levels are detected in urine for bladder cancer diagnosis and prognosis. Intravesical MIF inhibitors are a potential new treatment for bladder cancer. Blocking MIF in vitro attenuated bladder cancer cell proliferation, induced apoptosis and prevented the secretion of other cytokines associated with bladder cancer.

The invention has been disclosed broadly and illustrated in reference to representative embodiments described above. Those skilled in the art will recognize that various modifications can be made to the present invention without departing from the spirit and scope thereof.

1-79. (canceled)

80. A method for inhibiting proliferation of bladder carcinoma cells comprising the step of inhibiting macrophage migration inhibitory factor (MIF).

81. The method of claim 80, wherein excretion of macrophage MIF from bladder epithelia is inhibited.

82. The method of claim 80, wherein production of macrophage MIF by bladder epithelia is inhibited.

83. The method of claim 80, wherein the inhibition step is accomplished by an anti-MIF antibody.

84. The method of claim 80, wherein the inhibition step is accomplished by a MIF-antagonist.

85. The method of claim 80, wherein the inhibition step is accomplished by hylaluronan.

86. The method of claim 80, wherein the inhibition step is accomplished by anti-sense MIF oligonucleotides.
